(19) United States
(12) Patent Application Publication
(10) Pub. No.: US 2025/0354177 A1
(43) Pub. Date: Nov. 20, 2025

(54) SYSTEM AND METHODS FOR DUPLICATING TARGET FRAGMENTS

(71) Applicant: WUHAN UNIVERSITY, Wuhan, Hubei (CN)

(72) Inventors: Hao YIN, Wuhan, Hubei (CN); Ying ZHANG, Wuhan, Hubei (CN); Ruiwen ZHANG, Wuhan, Hubei (CN); Zhou HE, Wuhan, Hubei (CN)

(21) Appl. No.: 18/868,703

(22) PCT Filed: May 30, 2023
(86) PCT No.: PCT/CN2023/097095

§ 371 Date: Nov. 22, 2024

(30) Foreign Application Priority Data
May 30, 2022 (WO) PCT/CN2022/095910

(51) Int. Cl. C12N 15/90; C12N 9/12; C12N 9/22; C12N 15/11

(52) U.S. Cl. CPC C12N 15/907; C12N 9/1276; C12N 9/226; C12N 15/11; C12Y 207/07049; C12N 2310/20

(57) ABSTRACT

Provided are compositions and methods useful for duplicating/amplifying a target fragment on a target DNA sequence such as a genome sequence. The editing system employs a pair of pegRNA which, by virtue of their targeting sites flanking the target fragment, extend the target fragment with reverse transcriptase (RT) templates included in the pegRNA. As the two RT templates at least include portions that are complementary to each other, they can form a duplex region which can then serve as starting point for DNA polymerase to synthesize a new strand for each strand of the target fragment, thereby duplicating the target fragment. Continue this process introduce amplification of this targeted sequence. Alternatively, this process can be done by combination of pegRNA/sgRNA or sgRNA/sgRNA. In the case of sgRNA/sgRNA in a PAM-out position, the RT enzyme and templates are not required.

### BACKGROUND

Insertion of a nucleic acid fragment to a target nucleic acid, such as a genomic sequence, is on its own a challenging task. Targeted transgene integration is usually achieved by the homology-directed repair (HDR), which is inefficient in non-dividing cells and limited by the exogenous DNA donor. Homology-independent targeted integration (HITI) strategy has been developed to be independent of cell cycle. However, the efficiency of HITI remains low at genomic level (usually around 1-5%), and a mixed of integration events were observed.

A CRISPR-based gene editor, called Prime editing (PE) was recently developed through linking a reverse transcriptase (RT) to a Cas9 nickase. The RT template (RTT) is at the 3′ of the prime editing guide RNA (pegRNA), leading to precise modification of the nicked site. Prime editing is able to mediate all types of base editing, small insertion and deletion without donor DNA, holding great potential for basic research and correction of genetic mutants associated with human diseases. However, prime editing has not been used to insert larger fragment of DNA.

Duplicating a portion of the target nucleic acid, on the other hand, presents another challenge. In the prime editing technology, the inserted portion is primed by the RT template. Accordingly, the length being duplicated is limited. Also, each target portion for duplication would require a new RT template.

### SUMMARY

DNA sequence duplication has great potential for treating a variety of genetic diseases, and has great commercial value in industrial settings, such as protein production. There is currently no technology that can site-specifically amplify a fragment on a target DNA sequence, at least no such methodology based on the newly developed CRISPR technology.

The instant inventors developed a new method, termed Amplification Editing (AE) which is able to specifically amplify a target fragment on a target DNA sequence, such as a genomic sequence. An example AE method employs a pair of pegRNA which, by virtue of their targeting sites flanking the target fragment, extend the target fragment with reverse transcriptase (RT) templates included in the pegRNA. As the two RT templates at least include portions that are complementary to each other, they can form a duplex region which can then serve as starting point for DNA polymerase to synthesize a new strand for each strand of the target fragment, thereby duplicating the target fragment. When this process continues, the target fragment can be further amplified. Simply stated, a n-round amplification can generate up to 2ⁿ copies of the target fragment. At the same time, sequences encoded by the RT templates will be inserted between the duplicated target fragments.

According to one embodiment, the present disclosure provides a method for duplicating a target fragment of a target DNA sequence in the presence of a DNA polymerase, comprising contacting the target DNA sequence with (a) a Cas protein and a reverse transcriptase, (b) a first prime editing guide RNA (pegRNA) comprising a first CRISPR RNA (crRNA), and a first reverse transcriptase (RT) template sequence, and (c) a second prime editing guide RNA (pegRNA) comprising a second crRNA, and a second RT template sequence, wherein (i) the first RT template sequence comprises a first pairing fragment, (ii) the second RT template sequence comprises a second pairing fragment, (iii) the first pairing fragment and the second pairing fragment are complementary to each other, and (iv) the first pegRNA and the second pegRNA guide the Cas protein to cut, at two sites flanking the target fragment on the target DNA sequence, on opposite strands, thereby allowing (1) the reverse transcriptase to extend the two opposite strands of the target fragment, with the first and second RT template sequences as templates to generate two single-stranded flap DNA sequences, and (2) the two single-stranded flap sequences to form a double-stranded region allowing the DNA polymerase to extend the double-stranded region to duplicate each strand of the target fragment, thereby duplicating the target fragment, and inserting an inserted fragment between the two duplicated target fragments, wherein one strand of the inserted fragment comprises the first fragment, the first pairing fragment, and a reverse-complement of the second fragment.

In some embodiments, the first pegRNA further comprises a first primer-binding site (PBS) and a first spacer, and the second pegRNA further comprises a second PBS and a second spacer, enabling the pegRNA to guide the Cas protein to the two sites flanking the target fragment and to initiate reverse transcription.

In some embodiments, the first and second RT template sequences each is 0 to 2000 nucleotides long, preferably 15 to 500 nucleotide long.

In some embodiments, the first and second pairing fragments each is 0 to 1000 nucleotides long, preferably 3 to 200 nucleotides long or 3 to 50 nucleotides long, more preferably 30-100 nucleotides long.

In some embodiments, the first and second RT template sequences each further comprises a non-complementary template sequence not complementary to each other, wherein each non-complementary template sequence is located between the corresponding pairing fragment and crRNA, or between the corresponding pairing fragment and the PBS.

In some embodiments, each non-complementary template sequence is 1 to 2000 nucleotides long, preferably 1 to 1000 or 1 to 500 nucleotides long.

In some embodiments, the two sites flanking the target fragment are 2 to 1,000,000,000 base pairs apart, preferably 10 to 5,000,000 base pairs apart, from each other.

In some embodiments, each RT template sequence further comprises an extra sequence adjacent to the pairing fragment, wherein the two extra sequences are complementary to the target DNA sequence and have at least partial complementarity between each other.

Also provided is a method for duplicating a target fragment of a target DNA sequence in the presence of a DNA polymerase, comprising contacting the target DNA sequence with (a) a Cas protein, (b) a first single guide RNA (sgRNA) or tracrRNA, and (c) a second sgRNA or tracrRNA, wherein the first sgRNA or tracrRNA and the second sgRNA or tracrRNA each has sequence complementarity to a target site flanking the target fragment on the target DNA sequence, and the two target sites have at least partial complementarity between each other, wherein the first sgRNA or tracrRNA, in presence of the Cas protein, binds one strand and nicks the opposite strand of the first target site, releasing the opposite strand as a first single-stranded flap; wherein the second sgRNA or tracrRNA, in presence of the Cas protein, binds one strand and nicks the opposite strand of the second target site, releasing the opposite strand as a second single-stranded flap; and wherein the first single-stranded flap binds the second single-stranded flap to form a double-stranded region allowing the DNA polymerase to extend the double-stranded region to duplicate each strand of the target sequence, thereby duplicating the sequence between the two target sites.

In some embodiments, the partial complementarity includes complete complementarity for at least 3, 4, 5, 6, 7, or 8 consecutive nucleotides.

Yet also provided is a method for duplicating a target fragment of a target DNA sequence in the presence of a DNA polymerase, comprising contacting the target DNA sequence with (a) a Cas protein and a reverse transcriptase, (b) a prime editing guide RNA (pegRNA) comprising a first CRISPR RNA (crRNA), and a reverse transcriptase (RT) template sequence, and (c) a single guide RNA (sgRNA) or tracrRNA, wherein (i) the RT template sequence comprises a pairing fragment, (ii) the pegRNA guides the Cas protein to cut, at a first site proximate the target fragment on the opposite strand of the target DNA sequence, thereby allowing the reverse transcriptase to extend the opposite strand of the target fragment, with the RT template sequence as a template to generate a single-stranded flap DNA sequence, (iii) the sgRNA or tracrRNA guides the Cas protein to cut, at a second site proximate the target fragment on the opposite strand of the target DNA sequence, thereby releasing the opposite strand as a second single-stranded flap DNA sequence; and wherein the two single-stranded flap DNA sequences form a double-stranded region allowing the DNA polymerase to extend the double-stranded region to duplicate each strand of the target fragment, thereby duplicating the target fragment.

In some embodiments, the target DNA sequence is inside a cell. In some embodiments, the cell is a dividing cell. In some embodiments, the cell is not dividing.

In some embodiments, the target fragment is a telomere or a fragment thereof. In some embodiments, the method is carried out in vitro. In some embodiments, the method is carried out in vivo.

In some embodiments, the Cas protein is a nickase. In some embodiments, each pegRNA includes the first or second crRNA, the first or second pairing fragment, the first or second fragment, and the first or second PBS from 5′ to 3′ orientation. In some embodiments, the nickase is a Cas9 protein containing an inactive HNH domain which cleaves the target strand.

In some embodiments, the nickase is a nickase of SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9 or atCas9.

In some embodiments, the Cas protein is a Cas12 protein.

In some embodiments, each pegRNA includes the first or second crRNA, the first or second PBS, the first or second fragment, and the first or second pairing fragment, from 3′ to 5′ orientation.

In some embodiments, the Cas12 protein is Cas12a, Cas12b, Cas12f or Cas12i.

In some embodiments, the Cas12 protein is selected from the group consisting of AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, and LsCas12b.

In some embodiments, the first pegRNA or the second pegRNA further comprises a tail that (a) is able to form a hairpin or loop with itself, the PBS, the RT template sequence, the crRNA, or a combination thereof, or (b) comprises a poly(A), poly(U) or poly(C) sequence, or an RNA binding domain.

In some embodiments, the reverse transcriptase is M-MLV reverse transcriptase or a reverse transcriptase that can function under physiological conditions.

In some embodiments, the Cas protein and reverse transcriptase each is provided as a nucleotide encoding the respective protein, or as a protein.

In some embodiments, each pegRNA is provided as a recombinant DNA encoding the pegRNA, or as a RNA molecule.

In some embodiments, the duplicated target fragments, along with the inserted fragment are further duplicated.

### BRIEF DESCRIPTION OF THE DRAWINGS

FIG. 1 Overview of amplification editing. Schematic diagram of amplification editing (AE). FIG. 1a illustrates the structures of two pegRNAs that are in a PAM-out position (i.e., the genomic PAM sequences are not within the two sites targeted by the spacers). As shown in FIG. 1b, the target sites are nicked by nCas9-RT and reverse transcribed into two complementary 3′ flaps. These two 3′ flaps anneal to each other and serve as primers to initiate DNA replication. Then, the original genome DNA unwind and new DNA strands are synthesized. The new DNA strands stop synthesis when they meet the nick sites.

FIG. 2 Illustration of the experimental design to examine the AE results, and the results of the experiments. a, The diagram of three PCR methods to detect amplification editing. b, TAE agarose gel of PCR amplicons with methods in (a) shows 178 bp and 234 bp amplification with 20 bp insertion at VEGFA and HEK3 loci in HEK293T cells. c, Sanger sequencing of edit bands, which are pointed out by red triangle in Out-Out PCR column of (b) for VEGFA and HEK3 loci.

FIG. 3 Examine the effect of overlap and flap length in different loci. a-b, Duplication lengths are 178, 1014 and 7919 bp at VEGFA and C-MYC (b) loci in HEK293T cells. The editing efficiencies with flap and overlap from 10-100 bp were quantified by droplet digital PCR (ddPCR). c, The 3′ flaps are 30, 40, 50, 80 or 100 bp, and the overlap is 30 bp for duplication at VEGFA locus. d, the design fo ddPCR for quantify the efficiency of AE. The two sets of primers/probe designs for the control (reference genes/sequences): one targeted the same chromosome with at least 5 Kb from the duplication region, and the other targeted a different chromosome. The primers for the edited genotype were designed as the In-In PCR, and the probe was designed to target the duplicated sequence or inserted sequence.

FIG. 4 The effect of GC contents of the 3′ flap and multiplex editing for AE method. a, The GC contents of RTT are 30%, 50% or 80% for duplication at VEGFA and C-MYC loci. The duplication efficiencies were determined by ddPCR. Mean±s.d. of n=3 independent biological replicates. b, The efficiencies of AE by co-transfecting 2 or 3 pairs of pepRNAs targeting different regions were determined by ddPCR.

FIG. 5 The junction purity of AE method. a, Design of junction PCR. The PCR products were determined by next generation sequencing (NGS). b, The purities of junctions in control (WT) and edited genomes with 178 bp or 234 bp amplification at VEGFA and HEK3 loci by NGS. c, Junction purities using different flaps and overlaps for 178, 1014 and 7919 bp duplication were measured by NGS. d-e, Junction purities using various flaps and overlaps were measured by NGS for duplication at VEGFA (d) and C-MYC (e) loci in HEK293T cells.

FIG. 6 The efficiencies of AE at various loci in different cell lines. a-e, Efficiencies of AE at various loci with different amplification lengths in HEK293T cells (a), Huh-7 cells (b), K562 cells (c), U2OS cells (d) and N2a cells (e). Mean±s.d. of n=3 independent biological replicates.

FIG. 7 The edited outcomes of AE in various cell lines by TAE agarose. a-c, PCR amplicons of In-In and In-Out PCR were visualized by TAE agarose.

FIG. 8 Amplification editing for short sequences. a, Design of a 20 bp duplication. The length between two nicks is 20 bp, and two spacers are partially overlapped. b, Duplication efficiencies for short sequences. c, Analysis of reads from NGS of Out-Out PCR for 20 bp duplication.

FIG. 9 The tandem duplications by AE method. a, Overview of two models for multiple rounds of duplications by AE. Left: newly transcribed 3′ flaps are complementary to each other; they anneal to each other, and then new DNA strand is synthesized, resulting in products with 2ⁿ repeats after n rounds of duplication. Right: when the 3′ flap is paired and annealed with the inserted sequence, the products have [dup*(n−1)+x] amplified sequence (x<n), resulting in less repeats than 2ⁿ after n rounds of duplication. b, TAE agarose gel of Out-Out PCR amplicons from single cell colonies with various editing outcomes. A mixed outcomes within one colony are shown by triangle. c, Sanger sequencing of PCR amplicon in (b).

FIG. 10 ddPCR detection of editing efficiency with enzyme digestion. a, The amplification efficiencies of VEGFA-178 bp and RUNX1-1132 bp in K562 cells were quantified by ddPCR. b, Schematic diagram of restriction enzyme digestion of edited products for ddPCR. Without digestion, tandem repeated sequences of edited genome are in one droplet (left); After digestion, each repeat is in a different droplet (right). c-f, The duplication efficiencies at VEGFA and HEK3 loci in K562 (c, d) and HEK293T (e, f) cells were determined by ddPCR. Extracted genomic DNA was with or without a restriction enzyme treatment. Mean±s.d. of n=3 independent biological replicates. ***, P≤0.001.

FIG. 11 Restoration of EGFP function and duplication of a HBA gene using AE. a, The GFP sequence was disturbed and a 53 bp coding sequence was lost. GFP-L, N-terminal of GFP; GFP-R, C-terminal of GFP. b, AE treated cells were quantified by flow cytometry. c, Schematic diagram of generation of α-thalasemia genotype and restoration to normal genotype. Double-stranded breaks at the HBA1 (α1) and HBA2 (α2) genes were generated using Cas9 and two sgRNAs. It resulted in a 3.7 kb deletion and a fusion a gene (named as an), which is identical to al gene. AE was applied to correct this disease genotype to normal. d, The duplication efficiency of an was determined by ddPCR.

FIG. 12 Enhance the expression levels of miR-21 by AE. a, Amplification of the stem-loop (197 bp) in precursor of pre-miR-21 region. b, The efficiencies of miR-21 duplication at 3, 5 and 7 days in HEK293T and K562 cells. c, The expression levels of miR-21 were quantified by RT-qPCR. d, The expression levels of miR-21 targeting genes were determined by RT-qPCR. **, P≤0.01; *, P≤0.05. Mean±s.d. of at least 3 independent biological replicates.

FIG. 13 Duplication of large, megabase, and chromosomal scale DNA in HEK293T cells. a, Duplication of 30-100 Kb on chr 6 and 9 was determined by In-In PCR. Up: the PCR products were visualized by TAE agarose. Bottom: schematic diagram of Sanger sequencing for In-In PCR products. b, Copy numbers of IKZF4, RBMS2 and NEMP1 genes at chr 12 in control (WT) and 1 Mb duplicated cells from single cell colonies. c, Efficiencies of duplication of 30-100 Kb at chr 6 and 9 were quantified by ddPCR. d, Efficiencies of duplication of 1-3 Mb at chr 6, 9 and 12 were quantified by ddPCR. e, Efficiencies of duplication of 10-100 Mb at chr 6 and 9 were quantified by ddPCR. Mean±s.d. of n=3 independent biological replicates for c-f. ***, P≤0.001.

FIG. 14 Visualization the chromosomal scale duplication by AE in HEK293T cells. a-b, Fluorescence in situ hybridization (FISH) for 3 Mb duplication on chr 12 (a) and 100 Mb duplication on Chr 6 (b). Columns 1 (starting from left) represents chr 12 (a) or 6 (b) centromere. In columns 2, fluorescent probes label ˜108 Kb genomic sequence flanking STAT6 gene (a) or ˜198 Kb genomic sequence flanking ESR1 gene (b). Column 1 and 2 are merged in column 3. Column 4 is an enlarged view of column 3. In (a) or (b), Edit-1 and Edit-2 are different single cell colonies from AE treated samples for 3 Mb (a) or 100 Mb (b) duplication, respectively. The length unit for column 4 in (b) is micrometer.

FIG. 15 Exploring the shorter length of overlap. a, Efficiencies of duplication using 0-10 bp 3′ flap for VEGFA-178 bp amplification.

FIG. 16 Various PAM-out methods for amplification editing. a, Schematic diagram of different pegRNAs and sgRNAs combinations for amplification editing. Starting from the above to bottom, the first one (I) is the AE design we described above; the second one (II) is similar to the first one, but with part of the 3′ flap complementary to the genomic sequence near the nick site by the other pegRNA; the third one (III) applied one sgRNA and one pegRNA, whereas the 3′ flap generated by the pegRNA complementary to the genomic sequence near the nick site by the sgRNA; and the design in the bottom (IV) are duplication by two sgRNA and Cas9 nickase without RT enzyme, whereas the sequence of two nick sites are complementary to each other. b, Efficiencies of duplication using pegRNA+sgRNA or paired pegRNAs with 0, 8, 30 and 38 bp overlap at RUNX1, VEGFA and AAVS1 loci in HEK293T cells. c, The junction purities in (b) were quantified by NGS. d, TAE agarose gel of In-In PCR amplicons of paired sgRNA with 4 bp or 8 bp overlap at RUNX1, VEGFA and AAVS1 loci in HEK293T cells. e, The junction purities in (e) were quantified by NGS. ***, P≤0.001; **, P≤0.01; *, P≤0.05. Mean±s.d. of n=3 independent biological replicates.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### Amplification Editing

Prime editing (PE) is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

The pegRNA is capable of identifying the target nucleotide sequence to be edited, and encodes new genetic information that replaces the targeted sequence. The pegRNA consists of an extended single guide RNA (sgRNA) containing a primer binding site (PBS) and a reverse transcriptase (RT) template sequence. During genome editing, the primer binding site allows the 3′ end of the nicked DNA strand to hybridize to the pegRNA, while the RT template serves as a template for the synthesis of edited genetic information. Within the sgRNA portion, there are a spacer (guide sequence) that guides the prime editor to the target genomic site, and a sgRNA scaffold. When the guide sequence binds to the target genome sequence and dissociates the DNA double helix, the PBS binds to the opposite strand and initiates reverse transcription, using the RT template sequence as a template. The newly synthesized sequence (a “3′-flap”) ligates to the target genomic site, forming a double stranded DNA. The RT template can include mutations or small insertions relative to the target genome sequence, but needs to be largely homologous to the target genome sequence because the newly synthesized DNA strand should still be hybridized to one of the original target genome sequences.

The instant inventors designed and implemented a new technology, that is able to efficiently and specifically amplify a target fragment on a target DNA sequence, such as a genomic sequence. An example method employs a pair of pegRNA which, by virtue of their targeting sites flanking the target fragment, extend the target fragment with reverse transcriptase (RT) templates included in the pegRNA. As the two RT templates at least include portions that are complementary to each other, they can form a duplex region which can then serve as starting point for DNA polymerase to synthesize a new strand for each strand of the target fragment, thereby duplicating the target fragment.

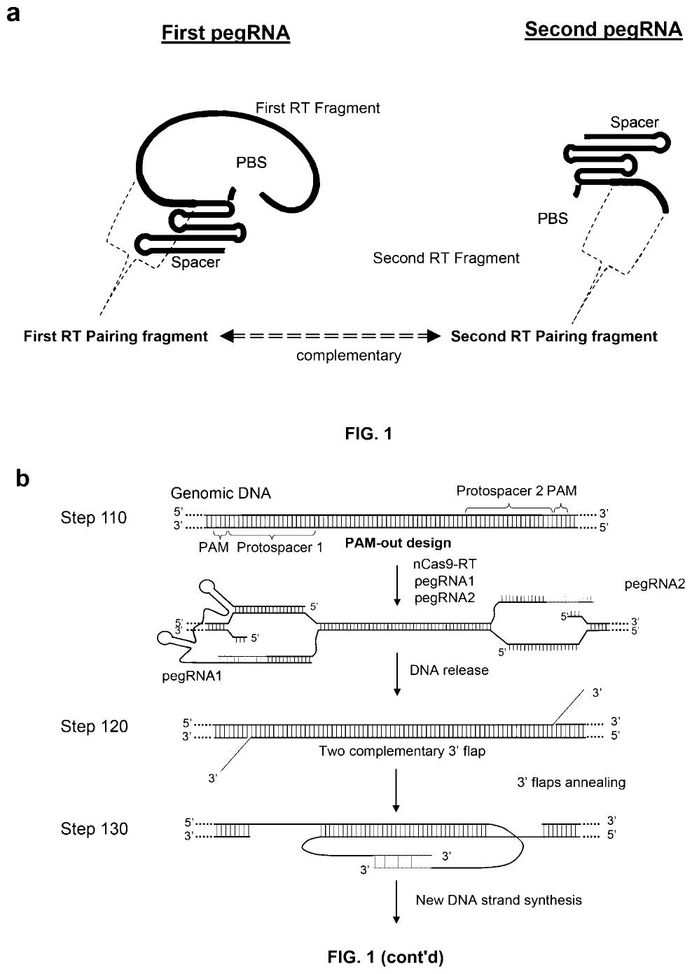

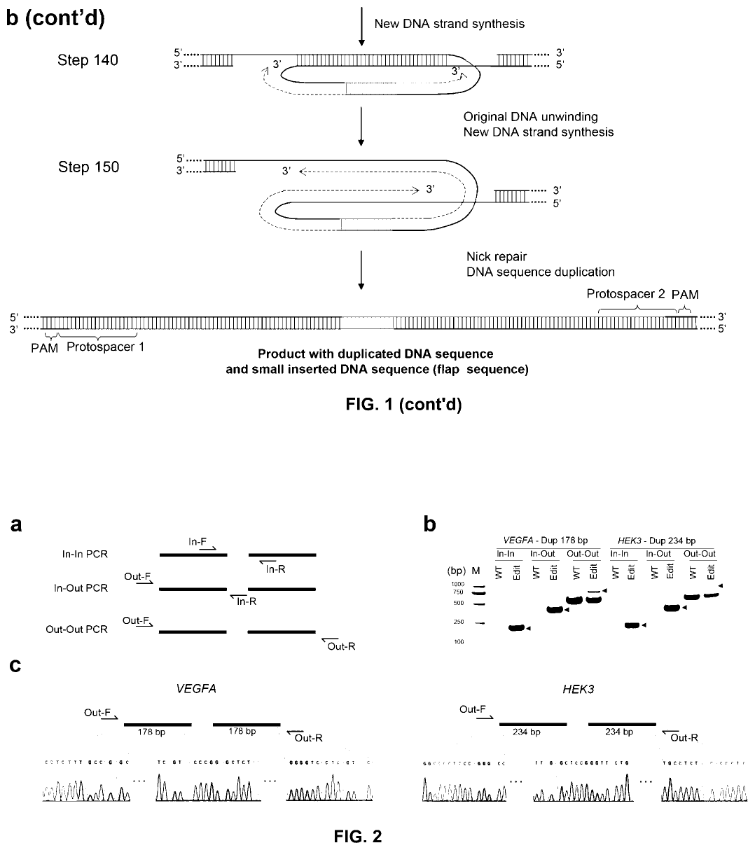

This new editing technology is termed “Amplification Editing” (AE), which is illustrated in FIG. 1. In an example AE procedure, two pegRNA molecules are employed. They are usually in the PAM-out position. Each of the pegRNA includes a CRISPR RNA (crRNA or sgRNA), a reverse transcriptase (RT) template sequence and a primer binding site (PBS). The PBS may be complementary to the guide sequence (or “spacer”) in the crRNA, but is typically a few nucleotides shorter. When the guide sequence binds to the target genome sequence and dissociates the DNA double helix, the PBS can bind to the opposite strand and initiate reverse transcription, using the RT template sequence as a template.

Unlike the pegRNA for the conventional prime editing, in each of the two pegRNA of the Amplification Editing system, the RT template sequence does not have to be homologous to the target genome sequence. In some embodiments, the RT template preferably has reduced or even no homology to the target genome sequence.

Instead, the two RT templates share a complementary portion. For instance, as illustrated in FIG. 1, in each pegRNA, the RT template includes two portions, a pairing RT fragment and a RT fragment. The two paring (complementary) RT fragments have complementary sequences, such that DNA sequences reverse transcribed from them can pair with each other.

It is noted, however, while the complementary portions are needed for forming a single-stranded DNA sequences that can bind to each other, the RT templates do not necessarily include the non-complementary “RT fragments.” In other words, in some embodiments, the two RT templates are entirely complementary to one another.

When the guide sequence binds to the target genome sequence and dissociates the DNA double helix, the PBS binds to the opposite strand and initiates reverse transcription, using the RT template sequence as a template. As shown in FIG. 1, the two pegRNA are designed with PAM-out design such that they will cut, on opposite strands, at two sites that flank a target portion to be duplicated (step 110). The RT templates then serve as templates to synthesize single-stranded DNA, and thereby introduce two single-stranded “flaps” extending duplication sequences, away from each other (step 120). The direction of the extension is ensured by the design of the pegRNA molecules.

Each flap includes a revere transcript from the RT fragment from the pegRNA and, more distal, one from the pairing (complementary) RT fragment. By virtue of their complementarity, these two distal fragments can hybridize with each other to form a duplex region (step 130). This duplex region is then able to serve as origin for DNA polymerase.

With the duplex region as origin and the single-stranded DNA of genome as templates, a new DNA strands is synthesized with original DNA unwinding between two nicks (steps 140-150), eventually the sequence of interest is precisely duplicated with a small inserted flap sequence in between (sequence generated by the 3′flap).

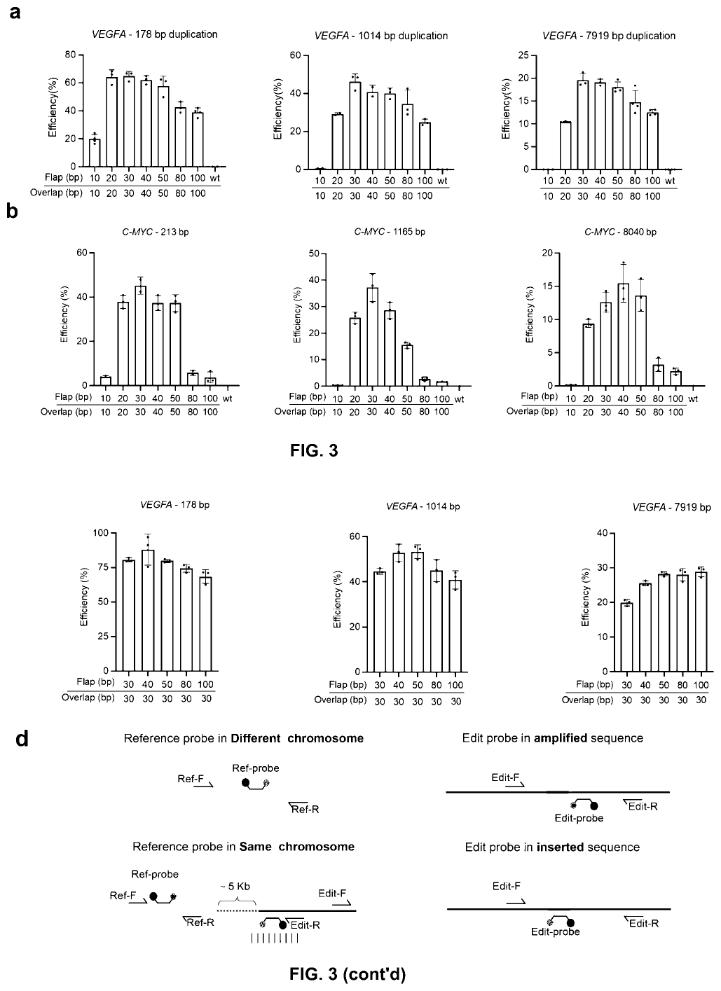

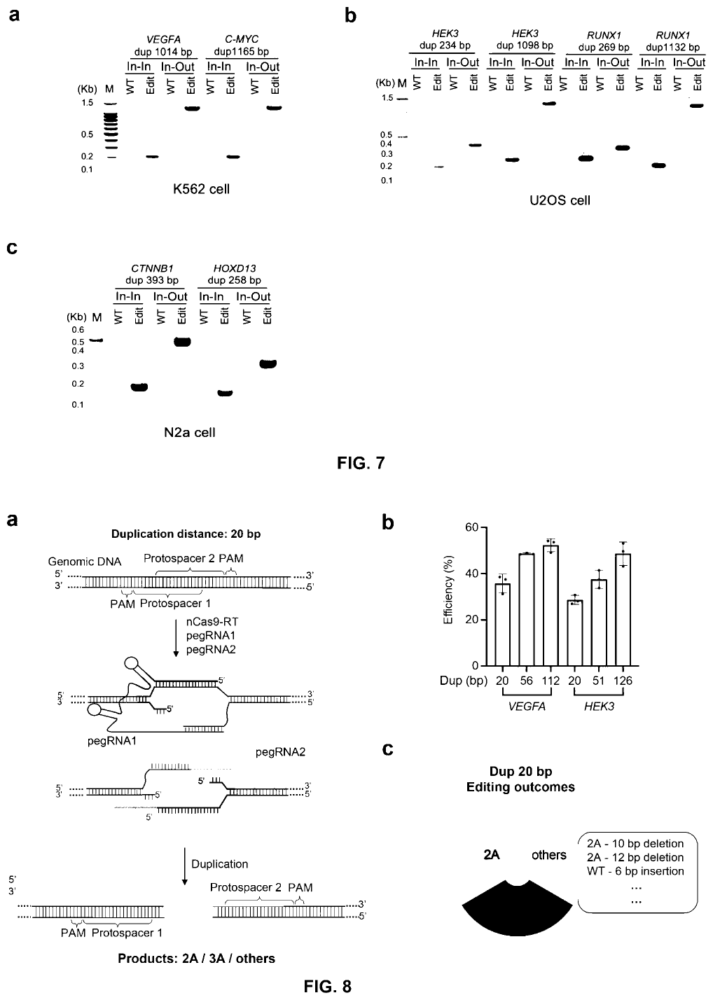

As demonstrated in Examples 1-2 (FIG. 2-3), this newly designed Amplification Editing system is highly accurate and efficient. Also, this new editing technology is so robust that the flap sequence can be as short as 10 nucleotides and as long as 100 nucleotides or more, without having a marked impact on the editing efficiency (Example 2, FIG. 3). AE is active in various cell lines and multiple genomic loci. (Example 3, FIG. 6-7). Also, AE could duplicate human genomic regions ranging from 20 bp to as large as at least 100 Megabase (Mb), which are very surprising, considering the average size of a human chromosome is 100 Mb scale (Example 2, FIG. 3, 8, 13-14).

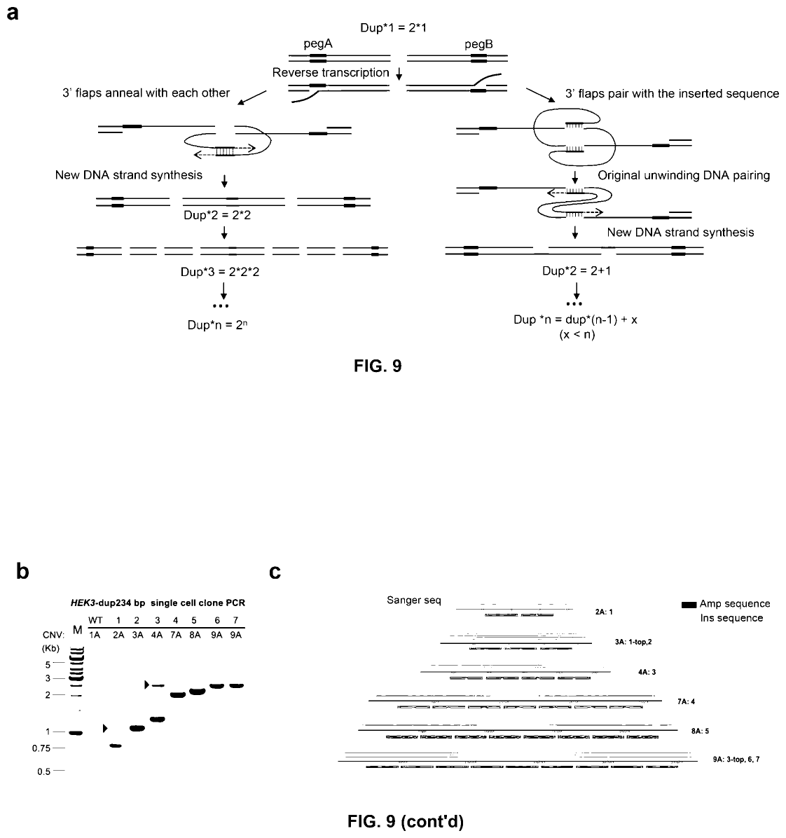

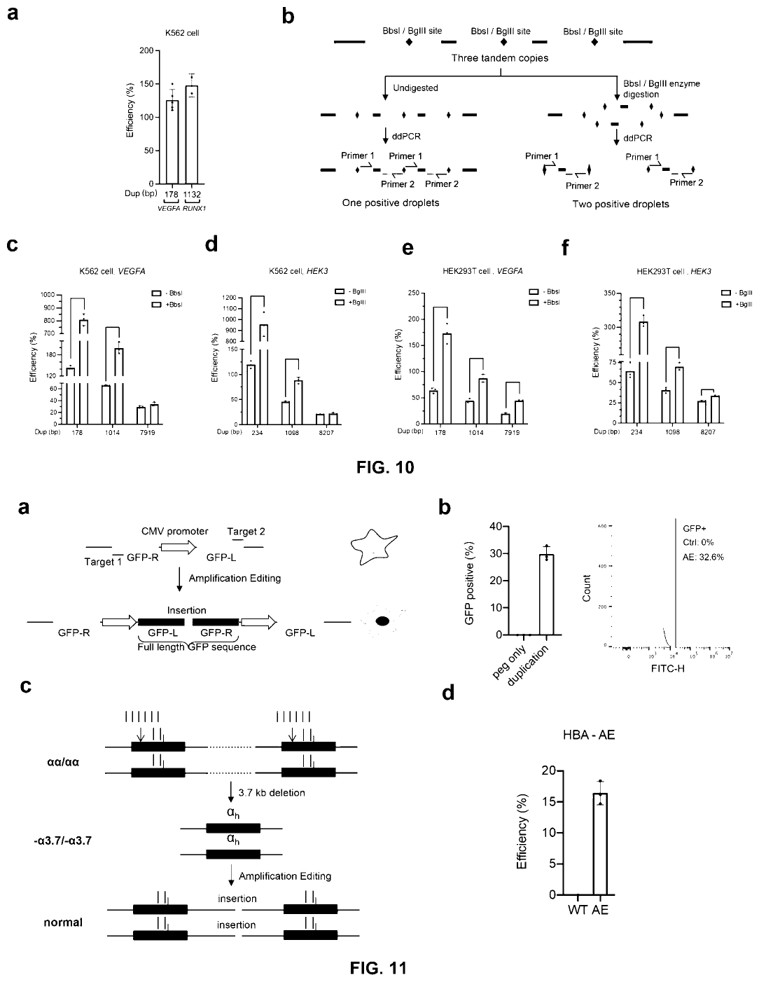

In yet another surprising discovery, the amplification can be recurring since each round of AE does not disrupt the flanking sequences that include the PAM sequences or nicking sites. As illustrated in FIG. 9 and demonstrated in FIG. 9-10, the AE method indeed was able to continue amplifying the target fragments. When newly transcribed 3′ flaps are complementary to each other; they anneal to each other, and then new DNA strand is synthesized, resulting in products with 2ⁿ repeats after n rounds of duplication. When the 3′ flap is paired and annealed with the inserted sequence, the products have [dup*(n−1)+x] amplified sequence (x<n), resulting in less repeats than 2ⁿ after n rounds of duplication.

### Alternative AE Designs

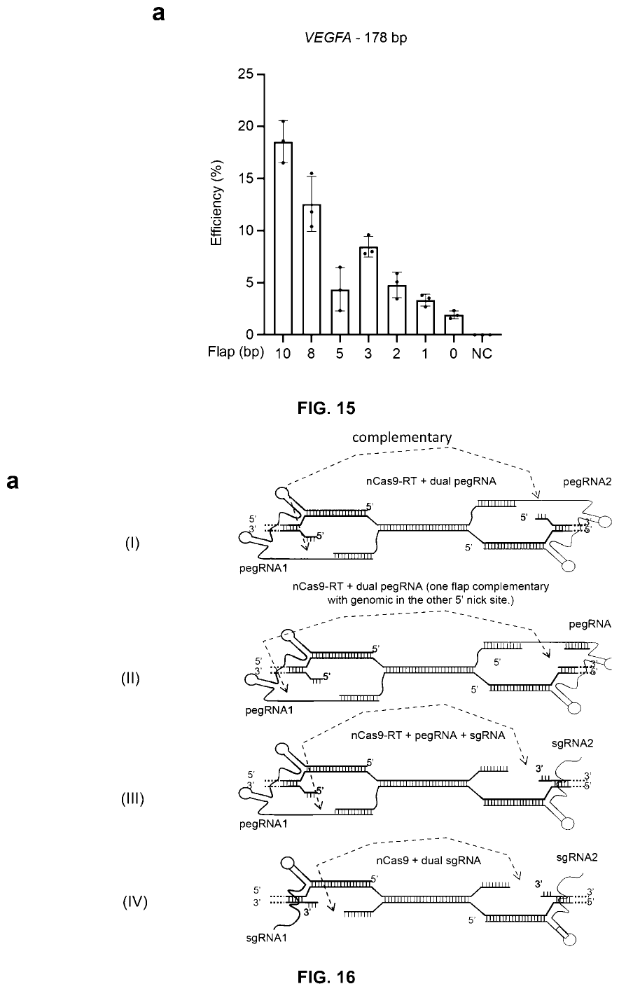

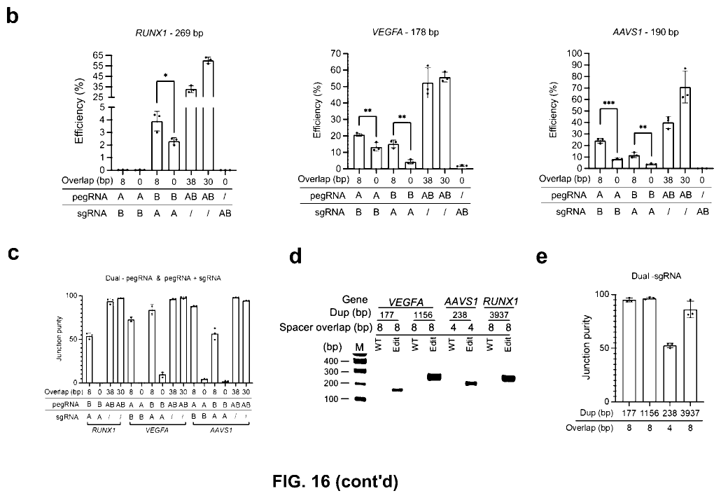

At the same time, an interesting phenomenon is that duplication can be achieved by methods other than paired pegRNAs. It has been achieved by using pegRNA/sgRNA (FIG. 16a, III) or sgRNA/sgRNA (FIG. 16a, IV) combinations, when these pairs were in PAM-out orientation and share complementary sequences near the nicking sites to allow annealing to form primers for DNA synthesis (FIG. 15-16). Alternatively, the 3′flap could be partially complementary to each other and meanwhile partially complementary to the genomic sequence near the nick site induced by the other pegRNA (FIG. 16a, II).

### pegRNA+sgRNA (or tracrRNA)

As demonstrated in Example 7, (FIG. 15-16), two complementary flaps are not required for the AE system. In design (III) of FIG. 16a, the assembly on the left still includes a nCas9-RT protein with a pegRNA, which can generate a 3′ flap following reverse transcription with the RT pairing fragment. On the right, however, only a conventional sgRNA (or conventional tracrRNA, i.e., without a RT template) is used, which can work with a conventional nCas9 protein, or the same nCas9-RT protein but whose RT activity is not required.

In this design, the single 3′ flap generated by the assembly on the left is complementary to a genomic sequence near the nicking site on the right. Their complementarity, as demonstrated in Example 7, can also initiate DNA unwinding and replication, leading to duplication of the sequence between the two sites.

### sgRNA+sgRNA (or tracrRNA+tracrRNA, sgRNA+tracrRNA, tracrRNA+sgRNA)

In yet another alternative design, a 3′ flap is not generated at all. Therefore, the entire system (FIG. 16a (IV)) does not include a reverse transcriptase or pegRNA. On both the left the right sides, a conventional nCas9/sgRNA (or tracrRNA) assembly is used, provided that two genomic sequences near the two nick sites are at least partially complementary to each other.

Without a single 3′ flap, the two sites, each with a nick, and exposing its non-targeted strand, allow annealing of the two complementary, non-targeted, genomic sequences and form primers for DNA synthesis, resulting in DNA replication.

### Hybrid pegRNA+Hybrid pegRNA

Another alternative of design (I) is design (II) (FIG. 16a). The design (II) still uses two nCas9-RT and two pegRNA sequences. Unlike design (I), however, the pegRNA of design (II) are partially complementary to each other, and partially complementary a genomic sequence near the nick site of the oppositive side.

In essence, in design (II), not only are the 3′ flap capable of initiating annealing, the adjacent genomic sequences can also play a part in it, allowing the 3′ flap sequences to be shorter.

In accordance with one embodiment of the present disclosure, therefore, provided is a method for duplicating a target fragment of a target DNA sequence in the presence of a DNA polymerase. In some embodiments, the method entails contacting the target DNA sequence with: (a) a Cas protein and a reverse transcriptase, (b) a first prime editing guide RNA (pegRNA) comprising a first CRISPR RNA (crRNA/sgRNA), a first reverse transcriptase (RT) template sequence, and (c) a second prime editing guide RNA (pegRNA) comprising a second crRNA/sgRNA, and a second RT template sequence. In some embodiments, the first pegRNA further comprises a first primer-binding site (PBS) and a first spacer, and the second pegRNA further comprises a second PBS and a second spacer.

In some embodiments, the first RT template sequence includes a first pairing fragment, the second RT template sequence includes a second pairing fragment, the first pairing fragment and the second pairing fragment are complementary to each other. Therefore, the first pegRNA and the second pegRNA can guide the Cas protein to cut, at two sites flanking the target fragment on the target DNA sequence, on opposite strands. Accordingly, the reverse transcriptase extends the two opposite strands of the target fragment, with the first and second RT template sequences as templates to generate two single-stranded flap DNA sequences.

Also, the two single-stranded flap sequences can form a double-stranded region allowing the DNA polymerase to extend the double-stranded region to duplicate each strand of the target fragment. Consequently, the target fragment is duplicated. Meanwhile, an inserted fragment is inserted between the two duplicated target fragments, wherein one strand of the inserted fragment comprises the first fragment, the first pairing fragment, and a reverse-complement of the second fragment.

In some embodiments, each RT template sequence further includes an extra sequence adjacent to the pairing fragment (the “hybrid pegRNA/hybrid pegRNA”), wherein the two extra sequences are complementary to the target DNA sequence and have at least partial complementarity between each other.

Yet another embodiment provides a method for duplicating a target fragment of a target DNA sequence in the presence of a DNA polymerase, comprising contacting the target DNA sequence with (a) a Cas protein, (b) a first single guide RNA (sgRNA), and (c) a second sgRNA, wherein the first sgRNA and the second sgRNA each has sequence complementarity to a target site flanking the target fragment on the target DNA sequence, and the two target sites have at least partial complementarity between each other, wherein the first sgRNA, in presence of the Cas protein, binds one strand and nicks the opposite strand of the first target site, releasing the opposite strand as a first single-stranded flap; wherein the second sgRNA, in presence of the Cas protein, binds one strand and nicks the opposite strand of the second target site, releasing the opposite strand as a second single-stranded flap; and wherein the first single-stranded flap binds the second single-stranded flap to form a double-stranded region allowing the DNA polymerase to extend the double-stranded region to duplicate each strand of the target sequence, thereby duplicating the sequence between the two target sites.

In some embodiments, the partial complementarity includes complete complementarity for at least 2, 3, 4, 5, 6, 7, or 8 nucleotides, which are preferably consecutive.

Also provided is a method for duplicating a target fragment of a target DNA sequence in the presence of a DNA polymerase, comprising contacting the target DNA sequence with (a) a Cas protein and a reverse transcriptase, (b) a prime editing guide RNA (pegRNA) comprising a first CRISPR RNA (crRNA), and a reverse transcriptase (RT) template sequence, and (c) a single guide RNA (sgRNA), wherein (i) the RT template sequence comprises a pairing fragment, (ii) the pegRNA guides the Cas protein to cut, at a first site proximate the target fragment on the opposite strand of the target DNA sequence, thereby allowing the reverse transcriptase to extend the opposite strand of the target fragment, with the RT template sequence as a template to generate a single-stranded flap DNA sequence, (iii) the sgRNA guides the Cas protein to cut, at a second site proximate the target fragment on the opposite strand of the target DNA sequence, thereby releasing the opposite strand as a second single-stranded flap DNA sequence; and wherein the two single-stranded flap DNA sequences form a double-stranded region allowing the DNA polymerase to extend the double-stranded region to duplicate each strand of the target fragment, thereby duplicating the target fragment.

The RT template sequences that encode the flap sequences can have different lengths without impacting the efficiency of the AE system. In one embodiment, each RT template sequence has a length of at least 1, 2, 3, 4, 5, 6, 7, 8, 9, 10, 15, 20, 25, 30, 35, 40, 50, 60, 70, 80, 90 or 100 nucleotides. In one embodiment, each RT template sequence has a length not longer than 10, 15, 20, 25, 30, 35, 40, 50, 60, 70, 80, 90, 100, 150, 200, 250, 300, 400, 500, 600, 700, 800, 900, 1000, 1500, or 2000 nucleotides. In some embodiment, each RT template sequence has a length of 3-2000, 10-2000, 10-500, 15-500, 15-200, 15-50 or 15-30 nucleotides.

As noted, the RT template sequences at least share a complementary portion (“pairing fragments”). In some embodiments, each pairing fragment has a length of at least 1, 2, 3, 4, 5, 6, 7, 8, 9, 10, 15, 20, 25, 30, 35, 40, 50, 60, 70, 80, 90 or 100 nucleotides. In one embodiment, each pairing fragment has a length not longer than 10, 15, 20, 25, 30, 35, 40, 50, 60, 70, 80, 90, 100, 150, or 200, or 500 nucleotides. In some embodiment, each pairing fragment has a length of 3-200, 5-200, 10-50, 10-25, or 15-20 nucleotides.

Optionally, besides the pairing fragment, each RT template sequence could also include a portion that does not have to be complementary to one another. In some embodiments, such a non-complementary template sequence is located between the corresponding pairing fragment and crRNA/sgRNA or located near the PBS sequence.

In some embodiments, the non-complementary template sequence has a length of at least 1, 2, 3, 4, 5, 6, 7, 8, 9, 10, 15, 20, 25, 30, 35, 40, 50, 60, 70, 80, 90 or 100 nucleotides. In some embodiments, the non-complementary template sequence has a length not longer than 10, 15, 20, 25, 30, 35, 40, 50, 60, 70, 80, 90, 100, 150, 200, 250, 300, 400, 500, 600, 700, 800, 900, 1000, 1500, or 2000 nucleotides. In some embodiment, each non-complementary template sequence has a length of 1-2000, 1-1000, 1-500, 10-500, 15-200, 15-50 or 15-30 nucleotides.

Optionally, besides the pairing fragment, each RT template sequence could or could not include a portion that is complementary to the genomic DNA near the nick site by the other pegRNA. In some embodiments, each pairing fragment with genomic DNA has a length of at least 1, 2, 3, 4, 5, 6, 7, 8, 9, 10, 15, 20, 25, 30, 35, 40, 50, 60, 70, 80, 90 or 100 nucleotides. In one embodiment, each pairing fragment has a length not longer than 10, 15, 20, 25, 30, 35, 40, 50, 60, 70, 80, 90, 100, 150, or 200, or 500 nucleotides.

The presently disclosed AE technology can amplify a target fragment of various length. In some embodiments, the target fragment has a length that is at least 10, 50, 100, 200, 500, 1000, 2000, 3000, 4000, 5000, 6000, 7000, 8000, 9000, 10000, 15000, 20000, 30000, 40000, 50000, 100,000, 1,000,000 (1 Mb) or 100,000,000 (100 Mb) or bp, or entire chromosome.

The length of the target fragment is defined by the two nicking sites flanking it. Accordingly, in some embodiments, the two sites flanking the target fragment are at least 10, 50, 100, 200, 500, 1000, 2000, 3000, 4000, 5000, 6000, 7000, 8000, 9000, 10000, 15000, 20000, 30000, 40000, 50000, 100000, 200000, 300000 1000000, 1000,000 or 100,000,000 bp apart or entire chromosome. In some embodiments, the two sites are less than 100, 200, 500, 1000, 2000, 3000, 4000, 5000, 6000, 7000, 8000, 9000, 10000, 15000, 20000, 30000, 40000, 50000, 200000, 300000 1000000, 1,000,000 or 100,000,000 bp apart. In some embodiments, they are 2 to 300,000 base pairs apart, preferably 10 to 50,000 base pairs apart, from each other.

The pegRNA disclosed herein can include other elements of conventional pegRNA as used in prime editing.

Prime editing is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

The pegRNA is capable of identifying the target nucleotide sequence to be edited, and encodes new genetic information that replaces the targeted sequence. The pegRNA consists of an extended single guide RNA (sgRNA) (or alternatively just a crRNA) containing a primer binding site (PBS) and a reverse transcriptase (RT) template sequence. During genome editing, the primer binding site allows the 3′ end of the nicked DNA strand to hybridize to the pegRNA, while the RT template serves as a template for the synthesis of edited genetic information. Within the sgRNA or crRNA portion, there are a spacer (guide sequence) that guides the prime editor to the target genomic site, and a sgRNA/crRNA scaffold.

In some embodiments, a pegRNA further includes a tail that (a) is able to form a hairpin or loop with itself, the PBS, the RT template sequence, the crRNA, or a combination thereof, or (b) comprises a poly (A), poly (U) or poly (C) sequence, or an RNA binding domain.

The Cas protein and the reverse transcriptase can be provided as a fusion protein, or separately but come together at the target site (e.g., as a complex). The fusion protein, in some embodiments, includes a Cas protein (e.g., nickase) fused to a reverse transcriptase. A nickase can be derived from a regular Cas9 protein, such as SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9 or atCas9. An example nickase is Cas9 H840A. The Cas9 enzyme contains two nuclease domains that can cleave DNA sequences, a RuvC domain that cleaves the non-target strand and a HNH domain that cleaves the target strand. The introduction of a H840A substitution in Cas9, through which the histidine residue at 840 is replaced by an alanine, inactivates the HNH domain. With only the RuvC functioning domain, the catalytically impaired Cas9 introduces a single strand nick, hence a nickase.

The conventional PE2 system is composed of Cas9 nickase-RT and pegRNA. The Cas12 proteins, however, have not been used in prime editing, primarily due to the lack of a corresponding Cas12 nickase. The conventional pegRNA is not expected to work with Cas12. A Cas9 nickase introduces a single-strand cut, but a Cas12 protein cuts both strands. A conventional pegRNA includes a single guide RNA (sgRNA) (or alternatively just a crRNA) which includes a spacer and a scaffold, a reverse transcriptase (RT) template sequence and a primer binding site (PBS), in a spacer-scaffold-RTT-PBS (5′ to 3′) configuration. If the target genome is cut in both strands by the Cas12 protein, the RTT in the pegRNA cannot serve as an effective RT template.

In a Cas9-based AE system, the RT template is flanked by the PBS and crRNA sequence. When a Cas12 protein is used, the RT template and the crRNA are placed on opposite sides of the PBS. In some embodiments, the nickase is a nickase of SpyCas9, SauCas9, NmeCas9, StCas9, FnCas9, CjCas9, AnaCas9, or GeoCas9.

The Cas protein may be a Cas12 protein, which may be Cas12a, Cas12b, Cas12f and Cas12i, without limitation. Examples include AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, and LsCas12b.

Non-limiting examples of reverse-transcriptases include human immunodeficiency virus (HIV) reverse-transcriptase, moloney murine leukemia virus (M-MLV) reverse-transcriptase and avian myeloblastosis virus (AMV) reverse-transcriptase, and any reverse transcriptases that can function under physiological conditions.

Amplification Editing can be carried out by transfecting target cells with the pair of pegRNAs or pegRNA/sgRNA and the fusion protein, or separated Cas9 nickase and reverse-transcriptases. Transfection is often accomplished by introducing vectors into a cell. In some embodiments, the editors can be introduced to a cell directly as proteins and RNA, or their complexes. Each molecule can be introduced separately, or together, without limitation.

Vectors may be introduced into the desired host cells by known methods, including, but not limited to, transfection, transduction, cell fusion, electroporation, and lipofection. Vectors can include various regulatory elements including promoters. In some embodiments, the present disclosure provides an expression vector including any of the polynucleotides described herein, e.g., an expression vector including polynucleotides encoding the fusion protein and/or the pegRNAs.

In some embodiments, the contacting occurs in a cell that includes a DNA polymerase. Such contacting can be, for instance, in a cell, in vitro, ex vivo, or in vivo. The cell may be a prokaryotic cell, a eukaryotic cell, a plant cell, an animal cell, a mammal cell, or a human cell.

In some embodiments, the cell is not actively dividing. In some embodiments, the cell is not actively undergoing cell division, or chromosome replication. In some embodiments, the cell is further engineered to express a DNA polymerase.

### Application of the AE Technology

DNA fragment amplification has broad applications in clinical and industrial settings.

For instance, duplication/amplification of some genes may lead to the occurrence of cancer. The AE method, therefore, can generate animal disease models (cells, mice, flies and other organisms), which are formed by gene duplication, and can be used to simulate this pathogenic information, for example, repeat expansion disorder and oncogene copy number variation.

Gene duplications can be useful in plants. Amplification of certain genes or genomic sequences (e.g., disease resistant genes) in plants have natural advantages of resistance to diseases, insect pests, and environmental stress. The AE technology, therefore, can help achieve these advantages for plants. Gene duplication could be useful in microorganism, for example, fungi, bacteria, or yeast, to produce gene cluster for gene clusters.

Certain diseases (α-globin deficiency) in humans are caused by haploid defects (haplo-insufficient). Duplication of such insufficient genes, therefore, can increase gene expression and restore patients to a normal phenotype. Chromosomal microdeletion is usually caused by 0.1 Mb to several Mb deletion in one chromosome copy. AE could amplify 0.1-10 Mb with high efficiency. AE could duplicate the corresponding sequences in the sister chromosome to compensate the gene loss due to microdeletion.

Therapeutic proteins have been produced in large volumes, such as vaccines and antibodies. The production efficiency may be limited by the number of copies of the coding sequence in a host cell (e.g., CHO cell), in particular the integrated ones. The AE technology can readily amplify such coding sequences, improving the yield of these proteins.

Cell cycles may be limited by the copy number/length of telomere in a cell. In one embodiment, a method is provided that uses the AE technology to amplify a telomere, or a portion therefore. Such amplification may increase the viability or replication ability of a cell, or increase the life span of an organism.

Compositions, kits and packages for such applications are also provided. In one embodiments, provided is a composition, kit or package for duplicating a target fragment of a target DNA sequence in the presence of a DNA polymerase, comprising: (a) a Cas protein and a reverse transcriptase, (b) a first prime editing guide RNA (pegRNA) comprising a first CRISPR RNA (crRNA), a first reverse transcriptase (RT) template sequence, and (c) a second prime editing guide RNA (pegRNA) comprising a second crRNA, and a second RT template sequence, wherein (i) the first RT template sequence comprises a first pairing fragment, (ii) the second RT template sequence comprises a second pairing fragment, (iii) the first pairing fragment and the second pairing fragment are complementary to each other, and (iv) the first pegRNA and the second pegRNA can guide the Cas protein to cut, at two sites flanking the target fragment, on opposite strands.

Also provided is a composition, kit or package for duplicating a target fragment of a target DNA sequence in the presence of a DNA polymerase, comprising (a) a Cas protein, (b) a first single guide RNA (sgRNA), and (c) a second sgRNA, wherein the first sgRNA and the second sgRNA each has sequence complementarity to a target site flanking the target fragment on the target DNA sequence, and the two target sites have at least partial complementarity between each other.

Yet further provided is a composition, kit or package for duplicating a target fragment of a target DNA sequence in the presence of a DNA polymerase, comprising (a) a Cas protein and a reverse transcriptase, (b) a prime editing guide RNA (pegRNA) comprising a first CRISPR RNA (crRNA), and a reverse transcriptase (RT) template sequence, and (c) a single guide RNA (sgRNA), wherein (i) the RT template sequence comprises a pairing fragment, (ii) the pegRNA can guide the Cas protein to cut, at a first site proximate the target fragment on the opposite strand of the target DNA sequence, thereby allowing the reverse transcriptase to extend the opposite strand of the target fragment, with the RT template sequence as a template to generate a single-stranded flap DNA sequence, (iii) the sgRNA can guide the Cas protein to cut, at a second site proximate the target fragment on the opposite strand of the target DNA sequence, thereby releasing the opposite strand as a second single-stranded flap DNA sequence.

Each of these elements is further described in the preceding sections, which are incorporated here.

### EXAMPLES

### Example 1. Design and Testing of Amplification Editors

A new prime editing (PE)-based system was designed to duplicate a target fragment on a target sequence. This new technology is termed “Amplification Editing” or AE. An example AE process is illustrated in FIG. 1a-b.

In the illustrated example, two pegRNA molecules are employed. With reference to FIG. 1a, each of the pegRNA includes, in addition to a CRISPR RNA (crRNA)/sgRNA, a reverse transcriptase (RT) template sequence and a primer binding site (PBS). The PBS may be complementary to the guide sequence (or “spacer”) in the crRNA/sgRNA, but is typically a few nucleotides shorter. When the guide sequence binds to the target genome sequence and dissociates the DNA double helix, the PBS can bind to the opposite strand and initiates reverse transcription, using the RT template sequence as a template.

Unlike the pegRNA for the conventional prime editing, in each of the two pegRNA of the Amplification Editing system, the RT template sequence does not have to be homologous to the target genome sequence. In some embodiments, the RT template preferably has reduced or even no homology to the target genome sequence.

Instead, the two RT templates share a complementary portion. For instance, as illustrated in FIG. 1a, in each pegRNA, the RT template includes two portions, a RT pairing fragment (proximate to the crRNA/sgRNA; or the “complementary portion”) and a RT fragment (distal from the crRNA/sgRNA) or otherwise in FIG. 16. The two paring (complementary) RT fragments have complementary sequences, such that DNA sequences reverse transcribed from them can pair with each other.

When the guide sequence binds to the target genome sequence and dissociates the DNA double helix, the PBS binds to the opposite strand and initiates reverse transcription, using the RT template sequence as a template. As shown in FIG. 1b, the two pegRNA are designed such that they will cut, on opposite strands, as “PAM-out” position at two sites that flank a target portion to be duplicated (step 110). The RT templates then serve as templates to synthesize single-stranded DNA, and thereby introduce two single-stranded “flaps” extending away from each other (step 120). The term “PAM-out” as used herein, refers to the placement of the PAM sequences being outside the region flanked by the two target sites (sites recognized by the spacers).

Each flap includes a revere transcript from the RT fragment from the pegRNA and, more distal, one from the pairing (complementary) RT fragment. By virtue of their complementarity, these two distal fragments can hybridize with each other to form a duplex region (step 130). This duplex region is then able to serve as origin for DNA polymerase.

With the duplex region as origin and the single-stranded genomic DNA as templates, a new DNA strand is synthesized with the original DNA unwinding between two nicks (steps 140-150), eventually replacing the target sequence to be duplicated (sequence A) with a new fragment that includes a first copy of Sequence A, an inserted portion based on the RT templates of both pegRNA, and a second copy of Sequence A. Essentially, the Amplification Editing process (A) duplicated Sequence A, and (B) inserted a new sequence based on the two RT templates between them.

This example further tested the Amplification Editing technology in the lab. We designed three types of PCR primers to examine the outcome of AE (FIG. 2a). The In-In PCR used a pair of primers for the target region but in the reverse direction, and it would not amplify the unedited sequence. The Out-Out PCR used a pair of primers outside the target region. The In-Out PCR use one primer outside the target region and another primer for the inserted sequence (generated by the 3′ flap).

We then used two pairs of pegRNAs, aiming to duplicate a 178 bp sequence in VEGFA locus and a 234 bp sequence in HEK3 locus in HEK293T cells. PCR bands were detected at expected size in AE edited cells using In-In PCR and In-Out PCR, but not in control cells, indicating a desired duplication (FIG. 2b). Two distinct bands were detected in AE edited cells using Out-Out PCR but only one band appeared in control cells, indicating the expansion of the target region (FIG. 2b). The large band from the Out-Out PCR was sanger sequenced, and the results showed the precisely duplicated sequence in the targeted loci with a 20 bp flap insertion in between (FIG. 2c).

### Example 2. Characterization and Optimization of AE

To quantify the efficiency of AE, droplet digital PCR (ddPCR) was applied. The primers for the edited genotype were designed as the In-In PCR, and the probe was designed to target the duplication region (FIG. 3d).

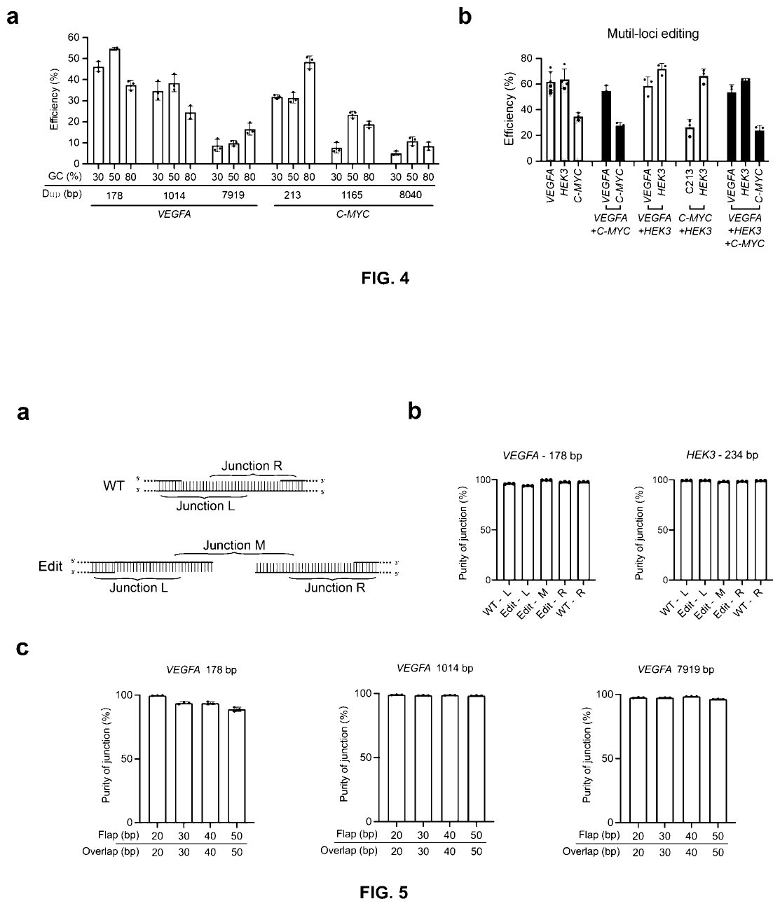

The length of the paired 3′ flap was designed from 10 bp to 100 bp, and they were all complementary to each other. Various duplicated sizes were examined for VEGFA and C-MYC loci. For duplication of ˜200 bp to ˜8 Kb size, the lengths of the 3′ flap ranged from 30-50 bp demonstrated high efficiency, with more than 60% duplication efficiency for a 178 bp duplication, more than 40% for 1 Kb duplication and ˜20% for 8 Kb duplication at VEGFA locus in HEK293T cells (FIG. 3a). The trend was similar for C-MYC locus (FIG. 3b). The pair of 3′ flap can be partially complementary (as “overlap”) to each other at their 3′ end. We used 30 bp overlap and extended the length of 3′ flap. Our data indicated that 30 bp 3′ flap showed high efficiency for ˜200 bp duplication, and 50 bp 3′ flap showed optimal efficiency for 8 Kb duplication (FIG. 3c). We also examined the effect of various GC content in the flap, and found that 30%-80% GC content all worked well for duplication (FIG. 4a). To explore the feasibility of simultaneous duplication in multiple loci, plasmids encoding two or three pairs of pegRNA were co-transfected into HEK293T cells. The duplication efficiencies for each locus via multiplex editing were comparable to single site editing (FIG. 4b).

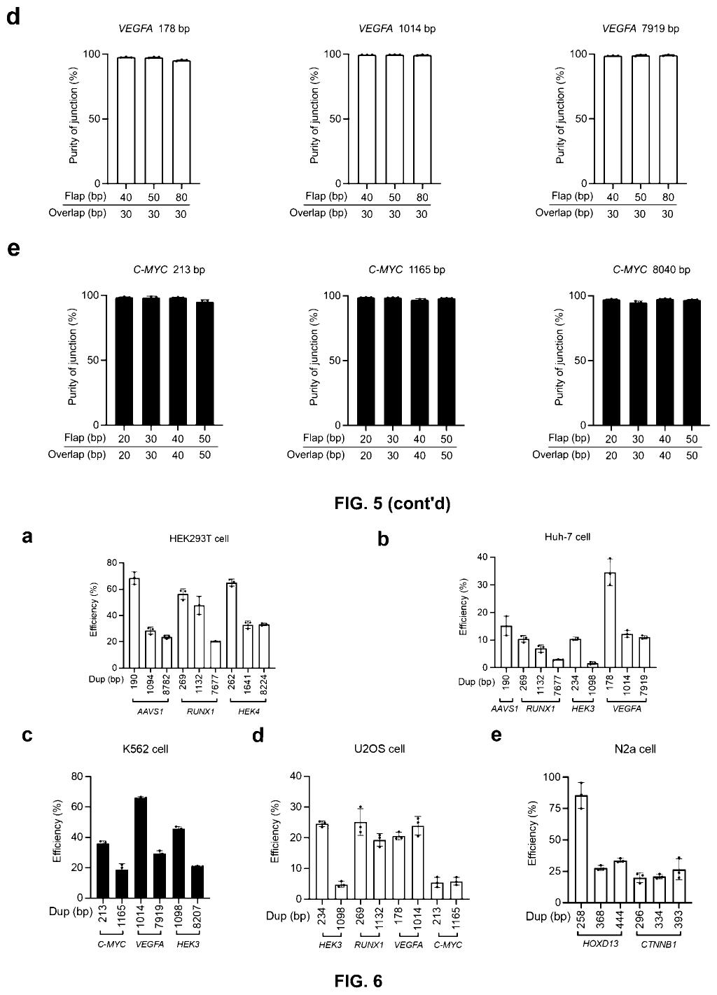

To determine the purity of the editing outcomes, we deep sequenced the left and right junction of the duplicated region, as well as the middle junction containing the flap insertion (FIG. 5a). The purities for all three junctions in VEGFA-178 bp duplication and HEK3-234 bp duplication were near 100% (FIG. 5b). The purities of junctions of ˜200 bp to ˜8 Kb duplication in VEGFA and C-MYC loci were all near 100%, indicating high precision of duplication by AE (FIG. 5c-e).

### Example 3. AE is Active in Multiple Cell Lines at Variously Endogenous Loci

AE has been examined in three endogenous sites above. We further expanded AE to duplicate other endogenous sites including AAVS1, RUNX1 and HEK4 and quantified the efficiency by ddPCR. We found that the duplication rate for ˜200 bp size ranged from 56.3% to 68.5%, ˜1-2 Kb size ranged from 28.5% to 47.8%, and ˜7-9 Kb size ranged from 20.4% to 33.3% in HEK293T cells (FIG. 6a).

Next, we examined whether AE was active in other cell lines such as human Huh-7 cells, human K562 cells, human U2OS cells and mouse N2a cells. Using ddPCR, we found that AE generated duplication frequencies of 1.7% to 34.6% for Huh-7 cells, 18.9% to 66.1% for K562 cells, 4.9% to 25.2% for U2OS cells, and 20.0% to 85.5% for N2a cells (FIG. 6b-e). We confirmed the duplication events in these cells using In-In and In-Out PCR (FIG. 7a-c).

### Example 4. AE Generates Tandem Duplications

We then explored whether AE could duplicate DNA size smaller than 150 bp. AE was designed for duplication between 20-130 bp, and its editing frequencies were from 28.6% to 52.3% (FIG. 8a-b). It is worth noting that when the duplication size is 20 bp, the spacers of two pegRNA are partially complementary to each other, and the primer binding site (PBS) overlap with the spacers of the other pegRNA (FIG. 8a). Therefore, the generation of each 3′ flap could be sequential. We deep sequenced the small size products from Out-Out PCR. The duplicated sequence was shown in the reads of deep sequencing as expected (FIG. 8c). It was not surprising that duplication with a small deletion was also detected, likely because two nicks were too close to each other for a 20 bp duplication. Interestingly, we identified three tandem repeats composed of 20 bp sequences interspaced with the flap insertion, suggesting that duplication can happen more than once (FIG. 8a-c, FIG. 9a).

We used Out-Out PCR to amplify the region of 234 bp duplication in HEK3 locus of single cell colonies. The PCR products shown in gel electrophoresis suggest the tandem repeats ranged from two to nine, and the sequence of these repeats were validated by sanger sequencing (FIG. 9b-c). It is possible that higher number of tandem repeats happened, but the length could be beyond the ability of genomic PCR. After one duplication, the recognition site for each pegRNA is still intact, enabling the next round of duplication. When the mechanism of the next round duplication is same as the first round, it generates 2n number of repeats (2A, 4A, 8A) (left scenario of FIG. 9a). Alternatively, the 3′ flap can pair with the insertion fragment from last round of duplication, to generate varied number of repeats (right scenario of FIG. 9a).

Consistent with the observation above, we identified more than 100% duplication efficiencies compared to the reference gene (reference probe) by ddPCR at VEGFA and RUNX1 loci in K562 cells (FIG. 10a). To determine the efficiencies of continuous duplication by AE, we fragmented the resulting tandem repeats using restriction enzymes. The fragmentated repeats from continuously duplicated samples would fall into different droplets of ddPCR to generate higher efficiency than the same sample without fragmentation (FIG. 10b). In contrast, samples with one duplication event would show no difference with or without fragmentation. The VEGFA-178 bp in K562 cells exhibited 808.7% efficiencies, and the VEGFA-1 Kb increased from 66.1% to 199.0% by ddPCR after fragmentation, in comparison to a reference gene (FIG. 10c). Similar data were obtained at HEK3 locus in K562 cells (FIG. 10d). The same trend was observed in HEK293T cells with duplication of ˜200 bp and 1 Kb size (FIG. 3f-g). When the duplication size reached 8 Kb, no difference was observed with or without fragmentation in K562 cells, and there was significant but small increase in HEK293T cells after fragmentation (FIG. 10e-f). These data indicate that continuous duplication is less frequent with larger duplication size.

### Example 5. Functional Assays and Potential Application of AE

To demonstrate AE can restore gene expression by duplication, we generated a stable cell line in which the GFP sequence was disturbed with a small deletion (53 bp). A pair of pegRNAs with PAM-out design was used to duplicate the GFP region and insert the lacking fragment, in order to restore the GFP expression (FIG. 11a). AE successfully generated ˜30% GFP positive cells via gene duplication (FIG. 11b).

Alpha-thalassemia is a common blood disorder involving mutations in two nearly identical genes HBA1 and HBA2. The most common form of alpha-thalassemia is the 3.7 Kb deletion of HBA1 and HBA2 genes (−α3.7), resulting in a fusion HBA gene, which is identical to HBA1 gene²⁸. We created −α3.7 genotype in HEK293T cells using CRISPR, and applied AE to duplicate the fusion HBA gene, to correct the −α3.7 deficiency (FIG. 11c). The efficiency of duplication for HBA gene reached 16.4% (FIG. 11d).

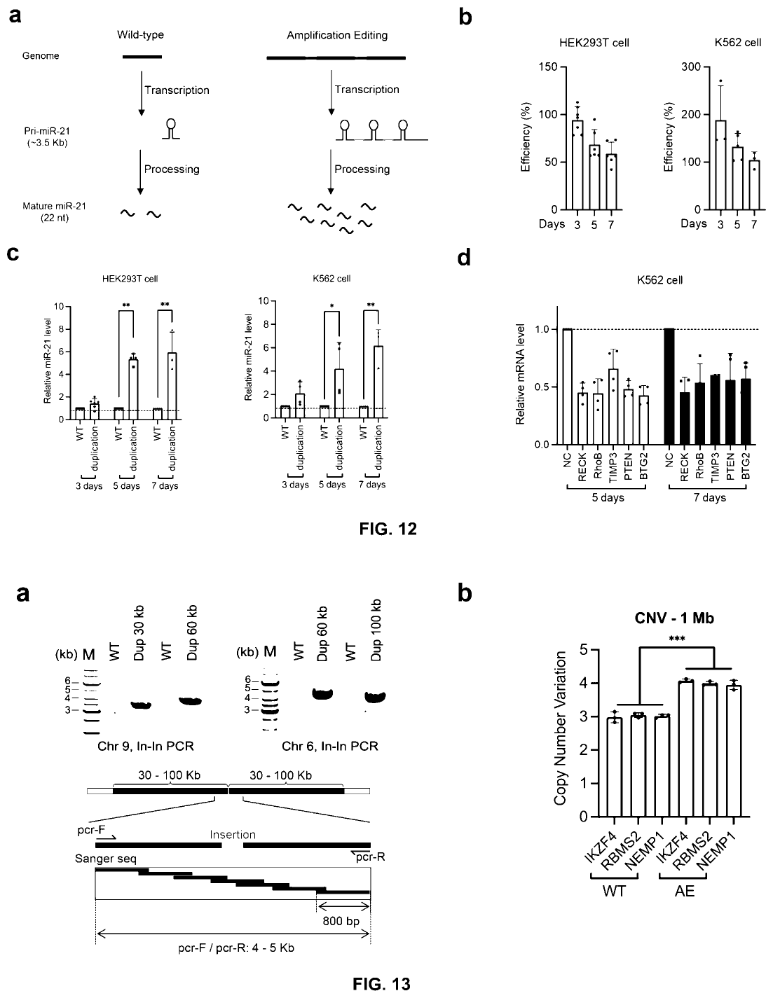

To demonstrate that AE treatment could cause functional changes of endogenous genes, we applied AE to amplify the stem-loop region of microRNA-21 (miR-21) (FIG. 12a). The duplication efficiencies were 94.6% and 189.0% in HEK293T and K562 cells, respectively, indicating multiple rounds of amplification for the stem-loop region (FIG. 12b). Compared to control cells, the expression levels of miR-21 significantly increased 5.9 and 6.2 folds in HEK293T and K562 cells 7 days after AE treatment, respectively (FIG. 12c). Correspondingly, the expression levels of miR-21 targeting genes decreased, indicating the duplication is functional (FIG. 12d).

### Example 6. AE can Duplicate 30 Kb to 100 Mb

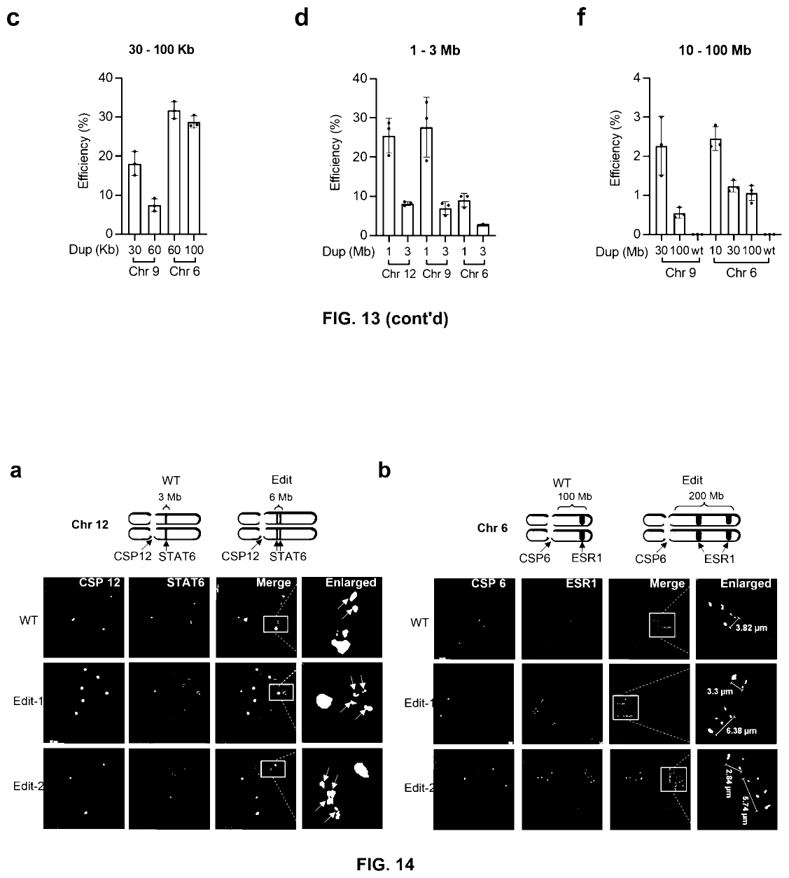

We explored whether AE could duplicate large sequences at a scale of 30 Kb-100 Mb. By designing a pair of pegRNAs spacing from 30, 60 and 100 Kb in Chromosome (Chr) 6 or in Chr 9, we performed In-In PCR to indicate the presence of duplication events. We examined multiple primers for In-In PCR in order to obtain as long as possible sequences. While control samples showed no bands by In-In PCR, the AE treated samples showed expected PCR products sized from 3.6 to 5.0 Kb (FIG. 13a). These PCR products were sanger sequenced using multiple primers, and the results indicated the duplication had correct sequence (FIG. 13a). The efficiencies of duplication determined by ddPCR ranged from 7.5% to 31.7% for 30-60 Kb duplication, and 28.8% for 100 Kb duplication (FIG. 13c).

Inspired by these results, we explored the possibility of genomic duplication by AE at Mb level. We first examined the efficiency to duplicate 1 and 3 Mb at Chr 6, 9 and 12. The duplication efficiencies of 1 Mb ranged from 9.0% to 27.7%, and 3 Mb ranged from 2.7% to 7.6% (FIG. 13d). To confirm the presence of Mb level duplication, we measured the CNV of three different genes in the duplicated region of Chr 12. The copy number of these genes in control cells were about 3, as triploid in this region. In single cell colonies from AE treated cells, the copy numbers were about 4, indicating duplication of this region in chr 12 (FIG. 13b).

We then explored the feasibility to duplicate 10 to 100 Mb. The efficiency for duplicating this chromosomal scale ranged from 0.55% to 2.5% at Chr 6 and Chr 9 (FIG. 13f). Notably, the size of Chr 6 is 172 Mb, and AE duplicated a 100 Mb region with 1.1% efficacy (FIG. 13f).

To confirm duplication of large region in the genome, we performed fluorescence in situ hybridization (FISH) using DNA probes targeting genomic sequences in the duplicated region. The STAT6 gene is located at the 3 Mb duplicated area of Chr 12. We used previously validated DNA probes targeting STAT6 gene to visualize this duplicated area, and DNA probes targeting centromeric region as the control (FIG. 14a). The wildtype cells showed two red dots surrounding a green dot, while single cell colonies of AE treated samples exhibited four dots surrounding a dot, indicating duplication of the STAT6 region (FIG. 14a). The 100 Mb duplicated region occupied most long arm (110 Mb) of Chr 6. We used validated DNA probes targeting ESR1 gene within this duplicated area of Chr 6 (FIG. 14b). The AE edited cells showed clearly elongated long arm (from 110 Mb in control to 210 Mb in edited cells) of Chr 6 with duplicated targeting region (FIG. 14b). These data collectively confirmed the presence of 1-100 Mb AE duplication of chromosomal arms in cells.

### Example 7. Various PAM-Out Methods for DNA Duplication

We explored in this example whether duplication could be achieved by methods other than paired pegRNAs. When a Cas9/sgRNA complex targets DNA, the guide sequence of sgRNA binds to its complementary sequence, leaving a free non-targeting DNA strand, as a small 3′ flap. We first examined the efficiencies of duplication with 10 bp or less complementary 3′ flap. The efficiency of duplication for 3 bp and 8 bp complementary 3′ flap was 8.5% and 12.6%, respectively (FIG. 15a). Interestingly, duplication efficiency was 1.9% in samples without any complementary 3′ flap formed by RTT of pegRNA, and this infrequent duplication is likely due to microhomology in the sgRNA binding site. It is possible to use pegRNA/sgRNA or sgRNA/sgRNA combinations, when these pairs are in PAM-out orientation and share complementary sequences to allow the 3′ flaps annealing (FIG. 16a). Alternatively, it was believed that a design with 3′ flaps partially complementary to the genomic sequence near the nick site by the other pegRNA, and partially complementary to each other is possible.

We determined the duplication efficiencies of pegRNA/sgRNA combinations, which were designed to have 8 bp complementary sequence between RTT of pegRNA and sgRNA targeting site, or without such deliberate design. While paired pegRNA showed 32.8%-71.0% duplication efficiencies in RUNX1, VEGFA and AAVS1 loci, the pegRNA/sgRNA combinations with 8 bp designed complementary sequence exhibited duplication efficiencies ranged from 3.9% to 24.3% (FIG. 16b). Although efficiencies of pegRNA/sgRNA without 8 bp complementary sequence were lower than those with this design, they also exhibited substantial editing, likely due to 3-4 bp microhomology between a different region of RTT and sgRNA targeting site (FIG. 16b). The junctions of duplication from pegRNA/sgRNA combinations were deep sequenced. The 8 bp complementary designed showed 53.9% to 87.9% purities, and samples without such design showed 0%-9.5% purities (FIG. 16c). Taking together, these data indicate the complementary design is key to maintain both the efficiency and precision of AE.

Next, we applied paired sgRNA in a PAM-out orientation with 4-8 bp complementary sequence in their targeting site near the nicks (FIG. 16a). The In-In PCR showed clear bands in paired sgRNA and Cas9 nickase treated samples, but not in control samples (FIG. 16d). The junction purity was up to 96.6% for 8 bp complementary sequences, indicating duplication with high precision could be achieved without nCas9-RT and pegRNA (FIG. 16e).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for duplicating a target fragment of a target DNA sequence in the presence of a DNA polymerase, comprising contacting the target DNA sequence with
(a) a Cas protein and a reverse transcriptase,
(b) a first prime editing guide RNA (pegRNA) comprising a first CRISPR RNA (crRNA), and a first reverse transcriptase (RT) template sequence, and
(c) a second prime editing guide RNA (pegRNA) comprising a second crRNA, and a second RT template sequence,
wherein (i) the first RT template sequence comprises a first pairing fragment, (ii) the second RT template sequence comprises a second pairing fragment, (iii) the first pairing fragment and the second pairing fragment are complementary to each other, and (iv) the first pegRNA and the second pegRNA guide the Cas protein to cut, at two sites flanking the target fragment on the target DNA sequence, on opposite strands,
thereby allowing (1) the reverse transcriptase to extend the two opposite strands of the target fragment, with the first and second RT template sequences as templates to generate two single-stranded flap DNA sequences, and (2) the two single-stranded flap sequences to form a double-stranded region allowing the DNA polymerase to extend the double-stranded region to duplicate each strand of the target fragment,
thereby duplicating the target fragment, and inserting an inserted fragment between the two duplicated target fragments, wherein one strand of the inserted fragment comprises the first fragment, the first pairing fragment, and a reverse-complement of the second fragment.

2. The method of claim 1, wherein the first pegRNA further comprises a first primer-binding site (PBS) and a first spacer, and the second pegRNA further comprises a second PBS and a second spacer, enabling the pegRNA to guide the Cas protein to the two sites flanking the target fragment and to initiate reverse transcription.

3. The method of claim 1, wherein the first and second RT template sequences each is 0 to 2000 nucleotides long, preferably 15 to 500 nucleotide long.

4. The method of claim 1, wherein the first and second pairing fragments each is 0 to 1000 nucleotides long, preferably 3 to 200 nucleotides long or 3 to 50 nucleotides long, more preferably 30-100 nucleotides long.

5. The method of claim 2, wherein the first and second RT template sequences each further comprises a non-complementary template sequence not complementary to each other, wherein each non-complementary template sequence is located between the corresponding pairing fragment and crRNA, or between the corresponding pairing fragment and the PBS.

6. The method of claim 5, wherein each non-complementary template sequence is 1 to 2000 nucleotides long, preferably 1 to 1000 or 1 to 500 nucleotides long.

7. The method of claim 1, wherein the two sites flanking the target fragment are 2 to 1,000,000,000 base pairs apart, preferably 10 to 5,000,000 base pairs apart, from each other.

8. The method of claim 1, wherein each RT template sequence further comprises an extra sequence adjacent to the pairing fragment, wherein the two extra sequences are complementary to the target DNA sequence and have at least partial complementarity between each other.

9. A method for duplicating a target fragment of a target DNA sequence in the presence of a DNA polymerase, comprising contacting the target DNA sequence with
(a) a Cas protein,
(b) a first single guide RNA (sgRNA) or tracrRNA, and
(c) a second sgRNA or tracrRNA, wherein the first sgRNA or tracrRNA and the second sgRNA or tracrRNA each has sequence complementarity to a target site flanking the target fragment on the target DNA sequence, and the two target sites have at least partial complementarity between each other,
wherein the first sgRNA or tracrRNA, in presence of the Cas protein, binds one strand and nicks the opposite strand of the first target site, releasing the opposite strand as a first single-stranded flap;
wherein the second sgRNA or tracrRNA, in presence of the Cas protein, binds one strand and nicks the opposite strand of the second target site, releasing the opposite strand as a second single-stranded flap; and
wherein the first single-stranded flap binds the second single-stranded flap to form a double-stranded region allowing the DNA polymerase to extend the double-stranded region to duplicate each strand of the target sequence, thereby duplicating the sequence between the two target sites.

10. The method of claim 9, wherein the partial complementarity includes complete complementarity for at least 3, 4, 5, 6, 7, or 8 consecutive nucleotides.

11. A method for duplicating a target fragment of a target DNA sequence in the presence of a DNA polymerase, comprising contacting the target DNA sequence with
(a) a Cas protein and a reverse transcriptase,
(b) a prime editing guide RNA (pegRNA) comprising a first CRISPR RNA (crRNA), and a reverse transcriptase (RT) template sequence, and
(c) a single guide RNA (sgRNA) or tracrRNA,
wherein (i) the RT template sequence comprises a pairing fragment, (ii) the pegRNA guides the Cas protein to cut, at a first site proximate the target fragment on the target DNA sequence, thereby allowing the reverse transcriptase to extend the opposite strand of the target fragment, with the RT template sequence as a template to generate a single-stranded flap DNA sequence, (iii) the sgRNA or tracrRNA guides the Cas protein to cut, at a second site proximate the target fragment on the target DNA sequence, thereby releasing the strand as a second single-stranded flap DNA sequence; and
wherein the two single-stranded flap DNA sequences form a double-stranded region allowing the DNA polymerase to extend the double-stranded region to duplicate each strand of the target fragment, thereby duplicating the target fragment.

12. The method of claim 1, wherein the target DNA sequence is inside a cell, which is optionally selected from the group consisting of a eukaryotic cell or a prokaryotic cell, a plant cell, an animal cell, a mammal cell, and a human cell.

13. The method of claim 12, wherein the cell is a dividing cell.

14. The method of claim 12, wherein the cell is not dividing.

15. The method of claim 1, wherein the target fragment is a telomere or a fragment thereof.

16. The method of claim 1, which is carried out in vitro.

17. The method of claim 1, which is carried out in vivo.

18. The method of claim 1, wherein the Cas protein is a nickase.

19. The method of claim 18, wherein each pegRNA includes the first or second crRNA, the first or second pairing fragment, the first or second fragment, and the first or second PBS from 5′ to 3′ orientation.

20. The method of claim 18, wherein the nickase is a Cas9 protein containing an inactive HNH domain which cleaves the target strand.

21-33. (canceled)
